(19) United States
(12) Patent Application Publication
(10) Pub. No.: US 2012/0018332 A1
(43) Pub. Date: Jan. 26, 2012

(54) Pressure Sensitive Construction for Patient Privacy Protection and Method of Using the Same

(75) Inventors: Anne M. DEHLINGER, North Tonawanda, NY (US); Blaine JACKSON, E. Aurora, NY (US); William BECKER, Lockport, NY (US); Rosalyn BEN-CHITRIT, Woodbridge, CT (US)

(73) Assignee: AVERY DENNISON CORPORATION, Pasadena, CA (US)

(21) Appl. No.: 13/160,546

(22) Filed: Jun. 15, 2011

Related U.S. Application Data
(60) Provisional application No. 61/354,873, filed on Jun. 15, 2010.

(51) Int. Cl. A61B 19/02; B42D 15/10; B65C 5/00; B65D 69/00; B65B 61/00

(52) U.S. Cl. 206/438; 206/570; 53/415; 283/74; 283/81

(57) ABSTRACT

The present invention relates to a pattern coated pressure sensitive label assembly for use in preserving patient privacy in medical treatment bag applications. The label assembly utilizes specific zones and areas to create removable sections that will permit areas containing patient information to be separated from the remainder of the label which remains with the treatment bag during disposal.

### CROSS-REFERENCE TO RELATED APPLICATION

The present application claims the benefit of U.S. Provisional Application No. 61/354,873 filed Jun. 15, 2010, which is incorporated herein by reference in its entirety.

### FIELD OF THE INVENTION

The present invention is found in the field of medical devices, more particularly to pressure sensitive information handling systems used in communication of patient and or treatment data for individuals undergoing one or more medical procedures.

### BACKGROUND OF THE INVENTION

The healthcare field today provides potential recipients and subscribers with a myriad of options and care choices. Many of the options require the capturing of patient or recipient information such as through the use of business forms, labels and the like which are then accumulated in various file assemblies to maintain a record of the particular transaction or treatment being contemplated.

With the increase in concern over the protection of personalized health care related data, various Federal and state regulations have been enacted requiring the care giver and participating organization to capture, retain and protect the information from dissemination without the patient's or individual's consent. The protection and retention of personal data can be even more problematic when one contemplates the amount of data being entered on individual record assemblies, treatment devices and the increase in the handling of such assemblies and devices. In addition, the efficiency of such transactions or treatment opportunities can be compromised if the data is not handled properly during use and disposal.

Government waste management regulations have been put in place, and the Environmental Protection Agency (EPA) announced that a mandatory survey be completed in 2009 in some 3500 facilities, including hospitals, long-term care facilities, hospices and veterinary facilities. There is a particularly serious concern in hospitals and other care facilities, due to the general lack of in-house expertise relating to medical and pharmaceutical waste disposal is preventing the care facility from reaching full compliance with waste management regulations.

In situations where a pharmacy staff may be aware of the correct disposal procedures and methods, in most if not many cases, such trained or experienced staff is simply not present when the caregiver or other staff member disposes of the residual drug waste. Communication of the information regarding the disposal of thousands of drugs is a challenge for them, especially as there are exceptions to disposal conditions depending on the amount of the residual drug is remaining in the container.

Failure to comply with the relevant waste procedures can subject the care facility with steep criminal and civil finds that could be as much as $37,500 per violation. This situation can of course be exacerbated when one considers the number of times or instances each day when pharmaceutical products, such as treatment bags are discarded. Pharmacists can also be held personally liable and face fines and imprisonment.

If a hospital, long term care facility, hospice or veterinary facility were to take a “better safe than sorry” attitude and simply dispose of all waste in the “hazardous waste” category or receptacle, the facility would then suffer from significant disposal costs associated with such procedures, e.g. heavy duty incineration, to dispose of such material thus, leading to a general need to increase the costs associated with providing health care to the general citizenry.

Another issue that must be addressed with the disposal and handling of pharmaceutical waste products arises out of HIPAA regulations which requires the maintenance of patient information in strict confidence and as such, medicines and treatment devices with patient information must not be thrown in the trash.

Disposal itself can be problematic if something other than incineration is selected as the disposal method. For example, the use of shredding equipment can become contaminated due to the build of plastics, adhesives of other residue which can require additional cleaning and lead to unscheduled maintenance in order to make sure that the equipment is operating in it most efficient manner.

What is needed therefore is a reliable method by which confidential patient data can be separated from the medical treatment device and disposed of in a secure manner to avoid liability and fines associated with recent legislative mandates.

### BRIEF SUMMARY OF THE INVENTION

The embodiments of the present invention described below are not intended to be exhaustive or to limit the invention to the precise forms disclosed in the following detailed description. Rather, the embodiments are chosen and described so that others skilled in the art may appreciate and understand the principles and practices of the present invention.

The invention is directed to pressure sensitive label assemblies that are intended for use in medical treatment bag applications, such as IV bags, plasma bags, pharmaceutical bags, nutritional supply bags and the like. The label assemblies provide a removable portion that includes patient information enabling the health care provider to preserve privacy of the patient when discarding the medical treatment bag.

In one exemplary embodiment, a pressure sensitive information handling assembly is provided and includes a substrate that has first and second faces, first and second longitudinally extending edges and first and second transversely extending sides. The first face has first and second areas, with the first area containing individualized information and the second area having a plurality of pre-determined waste disposal indicia.

The first area is surrounded by a series of perforations forming a line of weakness. The first area has a first edge aligned with the first longitudinally extending side, the first edge is free of perforations and the first area has a second edge inset from the second longitudinally extending side and having perforations to define the second edge. The first area has third and fourth edges each of which are parallel to and inset from the first and second transversely extending sides. The second face has first and second zones with the first zone provided with a pattern of adhesive and the second zone substantially free of adhesive. The second zone is in substantial juxtaposition with the first face first area.

In a still further embodiment of the presently described invention a pressure sensitive intermediate assembly is provided and includes a first sheet that has first and second faces with the first face having a release coated surface. A second sheet is disposed over the first sheet, with the second sheet including a plurality of separable label assemblies with each of the label assemblies having the construction set forth herein.

In a yet still further exemplary embodiment of the presently described invention, a pressure sensitive label assembly for use with a medical treatment bag is provided and includes a medical treatment bag having an outer surface and a pressure sensitive label assembly attached to the outer surface of the medical treatment bag, the pressure sensitive label assembly having the construction as set forth herein.

In a still further exemplary embodiment of the presently described invention, a method of using a pressure sensitive label assembly with a medical treatment bag is described and includes the steps of initially, providing a sheet of separable label assemblies having the construction as previously described herein and then printing patient information in the first area of the first face of one of the separable label assemblies to create a first printed label. Next, the first printed label is removed and is applied to a medical treatment bag. Once the medical treatment bag is used, the first area of the first printed label removed and each of the medical treatment bag and first area are disposed in separate disposal devices.

Other features and advantages of the present invention will become apparent to those skilled in the art from the following detailed description. It is to be understood, however, that the detailed description of the various embodiments and specific examples, while indicating preferred and other embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

These, as well as other objects and advantages of this invention, will be more completely understood and appreciated by referring to the following more detailed description of the presently preferred exemplary embodiments of the invention in conjunction with the accompanying drawings, of which:

FIG. 1 depicts a pressure sensitive label assembly produced in accordance with the present invention;

FIG. 1A shows the reverse side of the pressure sensitive label assembly of FIG. 1 showing the adhesive pattern;

FIG. 2 illustrates an intermediate assembly including a sheet of separable label assemblies for use in label medical treatment bags and the like;

FIG. 2A illustrates a further arrangement of an intermediate assembly;

FIG. 3 shows a pressure sensitive label assembly as described in the present invention used in connection with a medical treatment bag;

FIG. 4 provides a block diagram of an exemplary method of using a pressure sensitive assembly as described in the present invention;

FIG. 5 provides a pressure sensitive label assembly adhered to an object; and

FIG. 6 provides an illustration when the first area having indicia is un-adhered from the second area of the label.

### DETAILED DESCRIPTION OF THE INVENTION

The apparatuses and methods disclosed in this document are described in detail by way of examples and with reference to the figures. Unless otherwise specified, like numbers in the figures indicate references to the same, similar, or corresponding elements throughout the figures. It will be appreciated that modifications to disclosed and described examples, arrangements, configurations, components, elements, apparatuses, methods, materials, etc. can be made and may be desired for a specific application. In this disclosure, any identification of specific shapes, materials, techniques, arrangements, etc. are either related to a specific example presented or are merely a general description of such a shape, material, technique, arrangement, etc. Identifications of specific details or examples are not intended to be, and should not be, construed as mandatory or limiting unless specifically designated as such. Selected examples of apparatuses and methods are hereinafter disclosed and described in detail with reference made to FIGURES.

As indicated previously, the present invention is related to preserving the confidentiality of patient data, when it becomes necessary to associate such data with a treatment device such as a medical treatment bag or the like.

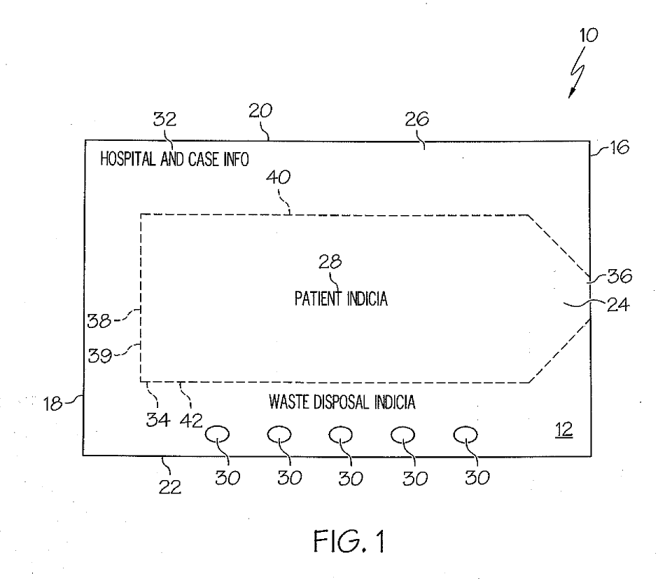

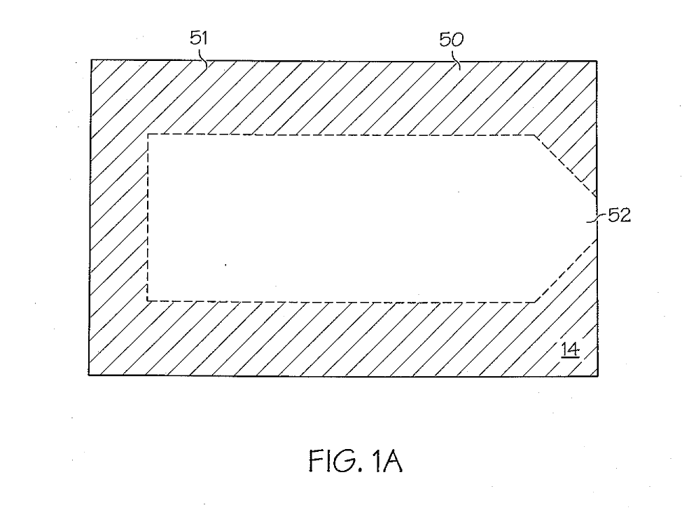

Reference is now directed to FIG. 1 of one embodiment of the presently described invention in which a pressure sensitive label assembly is generally depicted by reference 10. The label 10 has a first face 12 and a second face 14 (see FIG. 1A) first and second longitudinally extending sides 16, 18, respectively, and first and second transversely extending edges 20, 22 respectively.

The first face 12 of the label 10 is provided with first and second areas 24, 26, respectively. The first area 24, is shown as being substantially centered within the label 10 area, however it should be understood that the first area 24 may be situated anywhere within the label face 12.

The first area 24 contains individualized information 28, relating to the patient, treatment data and the like. The second area 26 has a plurality of pre-determined waste disposal indicia 30 that will indicate the required type of disposal, e.g. incineration, shredding, general trash, etc. Other indicia 32 may also be provided such as the hospital name, usage instructions and the like. The indicia 32 may be color coded in order to indicate a category for proper waste disposal.

The first area 24 surrounded by a series of perforations forming a line of weakness 34. The first area 24 has a first edge 36 aligned with the first longitudinally extending side 16. The first edge 36 is free of perforations and provides an area by which to begin removal of the first area 24 from the remainder of the label 10 as will be described herein. The first area 24 has a second edge 38 inset from the second longitudinally extending side 18 and has perforations 39 to define the second edge 38. The first area 24 has third and fourth edges 40, 42, respectively, each of which are parallel to and inset from the first and second transversely extending sides 20, 22, respectively.

Turning now to FIG. 1A, the second face 14 has first and second zones 50, 52, respectively, with the first zone 50 provided with a pattern of adhesive 51 and the second zone 52 substantially free of adhesive. The adhesive pattern 51 is generally “U” shaped and is preferably a permanent pressure sensitive adhesive. Additionally, the adhesive utilized in the present invention is plasticizer resistant as well as FDA 175.105 compliant. The adhesive should also be selected so as to not leach through or otherwise change the permeability of the bag to which is attached.

The second zone 52 is in substantial juxtaposition with the first face 12 first area 24. The second zone 52 may be provided with an opaque coating to further help conceal patient information from transferring to the underlying sheet.

In a preferred embodiment of the present invention, the label is constructed out of paper. However, it is contemplated by the present invention that the label may be made of other types of material.

As can be seen from each of FIGS. 1 and 1A the first edge 36 of the first area 24 is narrower than the second edges 38 of the first area as the line of weakness tapers toward the first longitudinally extending side 16.

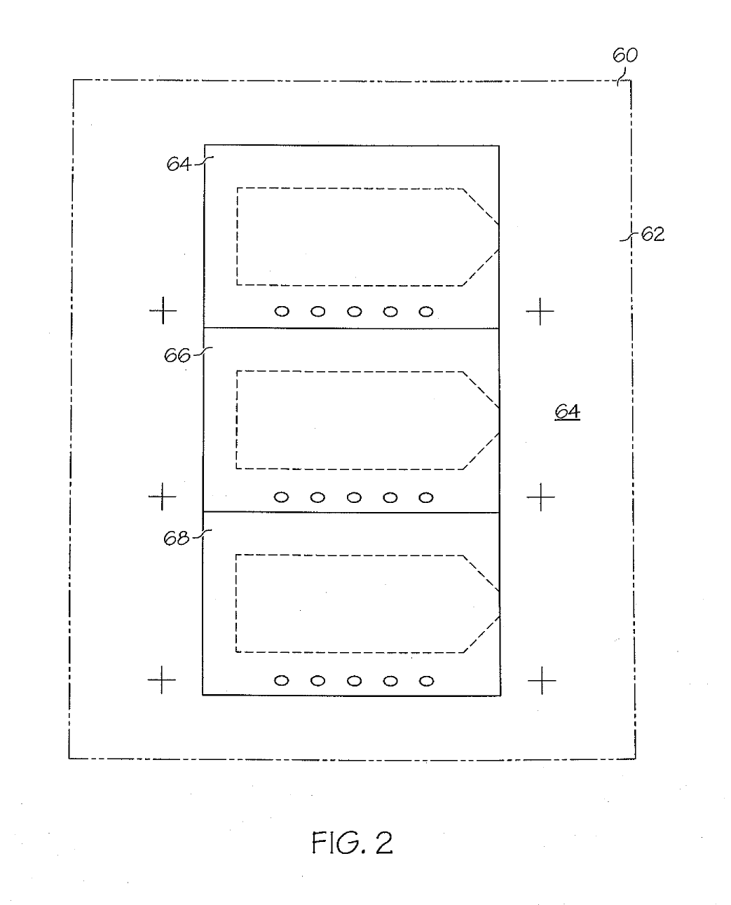

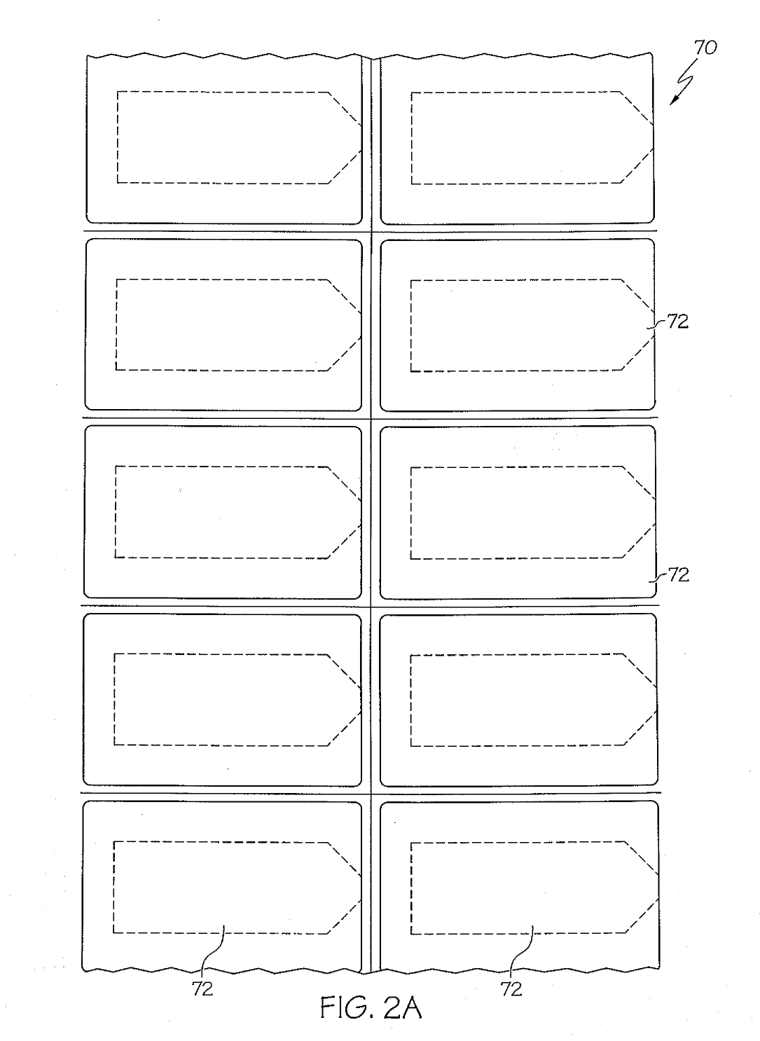

Attention is now directed to FIG. 2 of the presently described invention in which an intermediate assembly of labels 60 is provided. The assembly 60 includes a first sheet that has a first face and a second face (not shown) with the first face having a release coated surface. A second sheet 62 is disposed over the first sheet, with the second sheet 62 including a plurality of separable label assemblies 64, 66 and 68 with each of the label assemblies 64, 66 and 68 having the same construction as that provided in connection with the description of FIGS. 1 and 1A.

The assembly 60 has dimensions of roughly 8.5″ by 11″. Each of the label assemblies 62, 64 and 66 have dimensions of about 3 inches wide by about 4 inches long. Referring back to FIG. 1, the first edge 36 of the first area 24 has a length of about a half an inch and the second edge 38 of the first area 24 has a length of about 1.25 inches. The printable area of first area 24 is about 4 inches.

Reference is now directed to FIG. 2A which presents an alternate arrangement of a sheet 70 having a different configuration of removable labels 72. While the configuration provided in FIG. 2 is for cut sheet applications, such as desktop and sheet fed printers, the configuration of FIG. 2A is intended for example for a continuous thermal printer or other printers which may be used in connection with a continuous printing embodiment. It should however be understood that any configuration is possible and those provided in FIGS. 2 and 2A are intended for illustration purpose only.

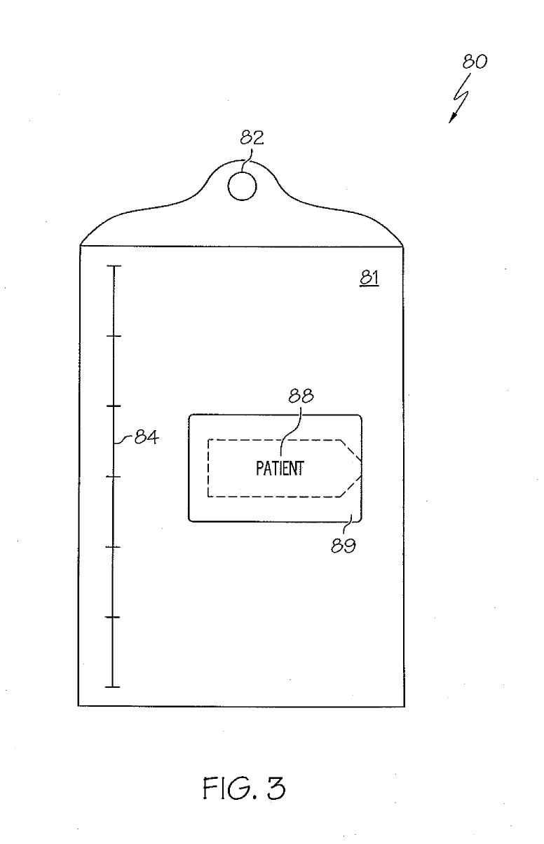

FIG. 3 provides an illustration of a medical treatment bag 80, such as an IV bag, plasma bag, pharmaceutical bag, nutritional supplement bag or the like. The bag 80 may be provided with a ring 82 so that the bag 80 may be suspended from a hook during treatment of the patient. The bag 80 may have other indicia 84 which may indicate fluid levels of the bag or the like. A pressure sensitive label assembly 86 is applied to the surface 81 of the bag 80. The label 86 has patient information printed in the removable section 88.

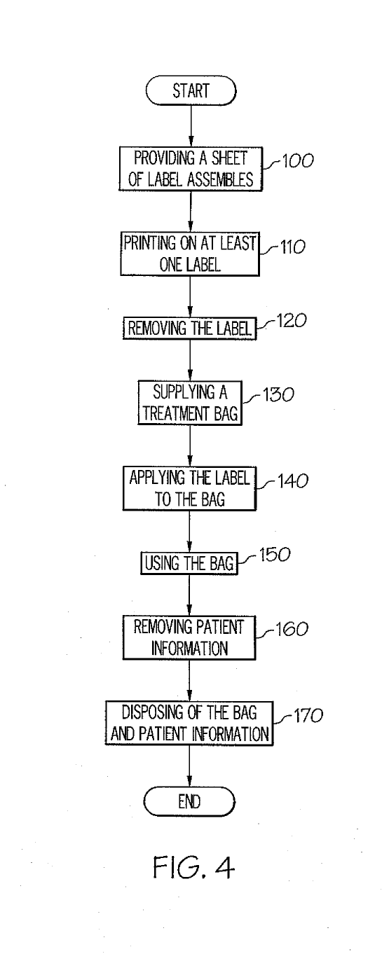

Attention is now directed to FIG. 4 which provides a block diagram of an exemplary method of using the pressure sensitive label assembly as described herein. The process is started by providing a sheet of label assemblies at step 100. The sheet of assemblies may be for example those depicted in connection with FIGS. 2 and 2A. Next, at step 110 printing of patient information is done in the first area of the first face of one of the separable label assemblies to create a first printed label. Next, the first printed label is removed at step 120 and is applied to a medical treatment bag at step 140 after the treatment bag is provided at step 130. Once the medical treatment bag is used or the contents of the bag are emptied or the treatment is over at step 150, the first area of the first printed label removed at step 160. This is accomplished by grabbing the first edge of the first area (see FIG. 1) and pulling off the first area along the line of weakness which was created by the perforations. Then, each of the medical treatment bag and first area are disposed in separate disposal devices at step 170 and the process ends. In this manner, the bag can be disposed of differently than the first area containing the patient information or they can be disposed in the same disposal area depending on the type of waste in the container. Disposal is determined through use of the waste disposal indicia on the label.

In one embodiment of the present invention, the second face 14 of the label 10 may have an additional block out layer in order to increase the label opacity.

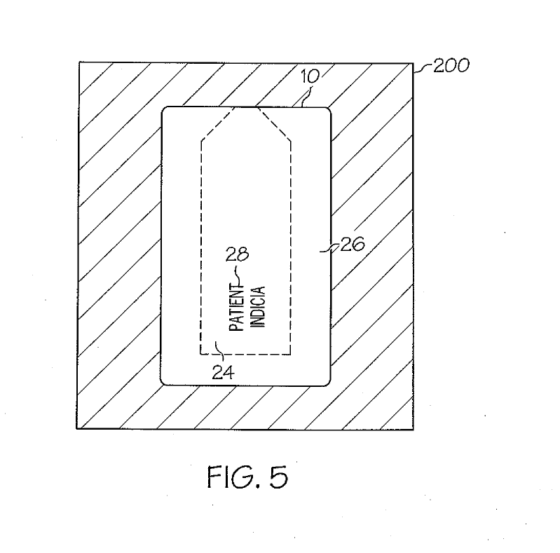

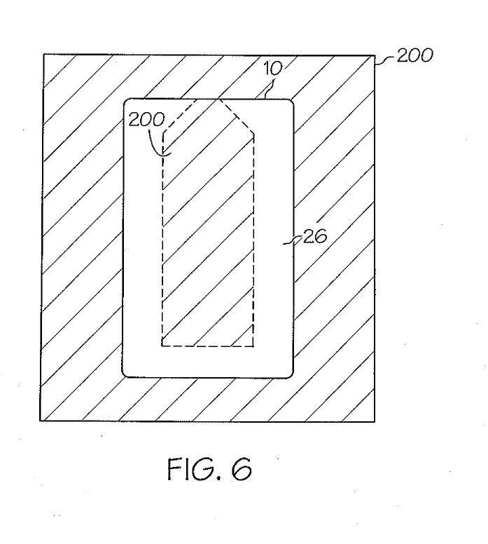

FIG. 5 illustrates when a label of the present invention is adhered to an object 200 such as an IV bag wherein the first area remains present within the label. FIG. 6, illustrates when the first area 24 is removed from the second area 26 leaving a portion of the object 200 visible where the first area 23 used to be and inside the perimeter of the second area 26. If a block out layer is adhered to the second face of the label 10, then a portion of the object will not be visible inside the perimeter of the second area 26. Instead a portion of the block out layer will be visible inside the perimeter of the second area 26.

Even though the label 10 of the present invention is most likely to be adhered to an IV bag, other applications are contemplated by the present invention. For example, the label of the present invention may be applied to files such as medical, and law files for identification purposes. Once the file does not need to be used anymore, the first area of the first printed label removed. The indicia 32 may be color coded for organizational, filing purposes. Additionally, labels of the present invention may be used for recycling purposes. The label can be added to a recyclable container for instance for identification purposes. Once the contents of the recyclable container are emptied, the first area of the label is removed, and the remaining second area having identifiable indicia may identify what category of recyclable material the container falls into i.e. paper or plastic.

It will thus be seen according to the present invention a highly advantageous pressure sensitive, information handling device for patient privacy applications has been provided. While the invention has been described in connection with what is presently considered to be the most practical and preferred embodiment, it will be apparent to those of ordinary skill in the art that the invention is not to be limited to the disclosed embodiment, and that many modifications and equivalent arrangements may be made thereof within the scope of the invention, which scope is to be accorded the broadest interpretation of the appended claims so as to encompass all equivalent structures and products.

The inventors hereby state their intent to rely on the Doctrine of Equivalents to determine and assess the reasonably fair scope of their invention as it pertains to any apparatus, system, method or article not materially departing from but outside the literal scope of the invention as set out in the following claims.

What is claimed is:

1. A pressure sensitive information handling assembly, comprising:
a substrate having first and second faces, first and second longitudinally extending edges and first and second transversely extending sides;
the first face having first and second areas, with the first area containing individualized information and the second area having a plurality of pre-determined waste disposal indicia;
the first area surrounded by a series of perforations forming a line of weakness, the first area having a first edge aligned with the first longitudinally extending side, the first edge is free of perforations, the first area having a second edge inset from the second longitudinally extending side and having perforations to define the second edge, the first area having third and fourth edges each of which are parallel to and inset from the first and second transversely extending sides; and
the second face having first and second zones with the first zone provided with a pattern of adhesive and the second zone substantially free of adhesive and the second zone is in substantial juxtaposition with the first face first area.

2. A pressure sensitive information handling assembly as recited in claim 1, wherein the first zone of the second face has a “U” shaped configuration.

3. A pressure sensitive information handling assembly as recited in claim 1, wherein the first edge of the first area tapers toward the first longitudinal side from the third and fourth edges of the first area.

4. A pressure sensitive information handling assembly as recited in claim 1, wherein the waste disposal indicia includes a plurality of colored marks corresponding to a waste categorization.

5. A pressure sensitive information handling assembly as recited in claim 1, wherein the first area is substantially centered within the first face.

6. A pressure sensitive information handling assembly as recited in claim 1, wherein the pattern of adhesive is a permanent pressure sensitive adhesive.

7. A pressure sensitive information handling assembly as recited in claim 1, wherein the second edge of the first area is longer than the first edge of the first area.

8. A pressure sensitive intermediate assembly, comprising:
a first sheet having first and second faces, the first face having a release coated surface;
a second sheet disposed over the first sheet, the second sheet including a plurality of separable label assemblies with each of the label assemblies including;
a substrate formed from a portion of the second sheet and having first and second faces, first and second longitudinally extending edges and first and second transversely extending sides;
the first face having first and second areas, with the first area containing individualized information and the second area having a plurality of pre-determined waste disposal indicia;
the first area surrounded by a series of perforations forming a line of weakness, the first area having a first edge aligned with the first longitudinally extending side, the first edge is free of perforations, the first area having a second edge inset from the second longitudinally extending side and having perforations to define the second edge, the first area having third and fourth edges each of which are parallel to and inset from the first and second transversely extending sides; and
the second face having first and second zones with the first zone provided with a pattern of adhesive and the second zone substantially free of adhesive and the second zone is in substantial juxtaposition with the first face first area.

9. A pressure sensitive intermediate assembly as recited in claim 8, wherein the first zone of the second face of each of the separable label assemblies has a “U” shaped configuration of permanent pressure sensitive adhesive.

10. A pressure sensitive intermediate assembly as recited in claim 8, wherein the first edge of the first area of each of the separable label assemblies tapers toward the first longitudinal side from the third and fourth edges of the first area and the second edge is larger than the first edge of the first area.

11. A pressure sensitive label assembly for use with a medical treatment bag, comprising:
a medical treatment bag having an outer surface;
a pressure sensitive label assembly attached to the outer surface of the medical treatment bag, the pressure sensitive label assembly including;
a substrate having first and second faces, first and second longitudinally extending edges and first and second transversely extending sides;
the first face having first and second areas, with the first area containing individualized information and the second area having a plurality of pre-determined waste disposal indicia;
the first area surrounded by a series of perforations forming a line of weakness, the first area having a first edge aligned with the first longitudinally extending side, the first edge is free of perforations, the first area having a second edge inset from the second longitudinally extending side and having perforations to define the second edge, the first area having third and fourth edges each of which are parallel to and inset from the first and second transversely extending sides; and
the second face having first and second zones with the first zone provided with a pattern of adhesive and the second zone substantially free of adhesive and the second zone is in substantial juxtaposition with the first face first area.

12. A pressure sensitive label assembly for use with a medical treatment bag as recited in claim 11, wherein the medical treatment bag includes IV bags, plasma bag, pharmaceutical bags, and nutritional bags.

13. The pressure sensitive label assembly of claim 11, further comprising a block out layer adhered to the second face of the label.

14. A method of using a pressure sensitive label assembly with a medical treatment bag, comprising the steps of:
providing a sheet of separable label assemblies, with each of the assemblies including a substrate having first and second faces, first and second longitudinally extending edges and first and second transversely extending sides;
the first face having first and second areas, with the first area containing individualized information and the second area having a plurality of pre-determined waste disposal indicia;
the first area surrounded by a series of perforations forming a line of weakness, the first area having a first edge aligned with the first longitudinally extending side, the first edge is free of perforations, the first area having a second edge inset from the second longitudinally extending side and having perforations to define the second edge, the first area having third and fourth edges each of which are parallel to and inset from the first and second transversely extending sides; and
the second face having first and second zones with the first zone provided with a pattern of adhesive and the second zone substantially free of adhesive and the second zone is in substantial juxtaposition with the first face first areal;
printing patient information in the first area of the first face of one of the separable label assemblies to create a first printed label;
removing the first printed label;
supplying a medical treatment bag;
applying the first printed label to the medical treatment bag;
using the medical treatment bag;
removing the first area of the first printed label; and
disposing of each of the medical treatment bag and first area in separate disposal devices.

15. The method as recited in claim 14, including a further step of encoding first printed label assembly with disposal indicia either simultaneously with or after the step of printing.

16. The method as recited in claim 14, wherein the step of removing is accomplished by separating the first area along the line of weakness.
